(19) United States
(12) Patent Application Publication
(10) Pub. No.: US 2014/0045220 A1
(43) Pub. Date: Feb. 13, 2014

(54) COMPOSITION FOR BIOSAMPLE TREATMENT AND METHOD FOR NUCLEIC ACID AMPLIFICATION USING THE SAME

(71) Applicant: Industrial Technology Research Institute, Hsinchu (TW)

(72) Inventors: Yi-Chang CHEN, Taipei City (TW); Jane S-C TSAI, Taipei City (TW)

(73) Assignee: Industrial Technology Research Institute, Hsinchu (TW)

(21) Appl. No.: 13/963,805

(22) Filed: Aug. 9, 2013

Related U.S. Application Data
(60) Provisional application No. 61/681,187, filed on Aug. 9, 2012.

(30) Foreign Application Priority Data
Dec. 28, 2012 (TW) 101150887

(51) Int. Cl. C12Q 1/68

(52) U.S. Cl. CPC C12Q 1/6848
USPC 435/91.2; 435/194; 252/182.12; 252/182.3; 252/182.31

(57) ABSTRACT

A composition for a biosample treatment and method for nucleic acid amplification using the same are provided. The composition for biosample treatment includes at least one halocarbon, at least one polyether and at least one surfactant. The composition contains 1˜70% by weight of the halocarbon based on the total weight of the composition. Accordingly, a biosample can be lysed and homogenized in a single tube at one step. Furthermore, reagents for use in nucleic acid amplification can be directly added in the same tube for nucleic acid amplification at the next step. The process, operation periods and risks of contamination can be therefore reduced and a result of nucleic acid amplification with less background noises can be therefore obtained as well.

### CROSS REFERENCE TO RELATED APPLICATIONS

This application claims the priority of Taiwan Patent Application No. 101150887, filed Dec. 28, 2012, which claims the priority of U.S. Provisional Application No. 61/681,187, filed Aug. 9, 2012, the disclosure of which is hereby incorporated by reference herein in its entirety.

### TECHNICAL FIELD

The technical field relates to a composition for biosample treatment and a method for nucleic acid amplification using the same.

### BACKGROUND

Current commercially available kits for use in nucleic acid amplification usually contain several reagents for different functions, such as reagents for sample treatments, like proteases or lysis buffers, and reagents for nucleic acid amplification, like polymerases, deoxyribonucleotides or buffers.

However, most of the commercially available reagent kits are multi-tube operated for nucleic acid amplification. Thus, the risk for sample contamination is high. In addition, the sensitivity of the final results is reduced due to the background noise from the solution used in the operation.

Therefore, novel reagents for nucleic acid amplification are demanded, which have less risk for sample contamination and less background noise.

### SUMMARY

One embodiment of the disclosure provides a composition for biosample treatment, which comprises at least one halocarbon, at least one polyether, and at least one surfactant. The composition contains 1˜70% by weight of the halocarbon based on the total weight of the composition.

Another embodiment of the disclosure provides a method for nucleic acid amplification, which comprises the following steps mixing a composition and a biosample to form a homogenized solution, mixing a reagent for nucleic acid amplification and the homogenized solution to form a mixture, and applying the mixture to nucleic acid amplification. The composition comprises at least one halocarbon, at least one polyether and at least one surfactant and contains 1˜70% by weight of the halocarbon based on the total weight of the composition.

### BRIEF DESCRIPTION OF THE DRAWINGS

The present invention can be more fully understood by reading the subsequent detailed description and examples with references made to the accompanying drawings, wherein:

FIG. 1 is a gel electrophoresis photograph showing PCR (polymerase chain reaction) amplification data of DNA samples treated with the composition according to one embodiment of the disclosure, compared to that treated with a commercially available reagent kit;

FIG. 2 is a gel electrophoresis photograph showing PCR data of DNA samples from several tissues treated with the composition according to one embodiment of the disclosure;

FIG. 3 is a plot showing real-time PCR (real-time polymerase chain reaction) data of 18S RNA amplification from blood samples treated with the composition according to one embodiment of the disclosure;

FIG. 4 is a plot showing real-time PCR data of GAPDH gene amplification from blood samples treated with the composition according to one embodiment of the disclosure;

FIG. 5 is a gel electrophoresis photograph showing RT-PCR (reverse transcription polymerase chain reaction) data of DNA samples from Dengue virus treated with the composition according to one embodiment of the disclosure, compared to that treated with a commercially available reagent kit;

FIG. 6 is a gel electrophoresis photograph showing multiplex PCR data of GAPDH gene, β-actin 01 gene and β-actin 02 gene amplification from sera samples treated with the composition according to one embodiment of the disclosure, compared to that treated with a commercially available reagent kit;

FIG. 7 is a gel electrophoresis photograph showing PCR data of several genes from plant tissues treated with the composition according to one embodiment of the disclosure;

FIG. 8 is a microscopic photograph (50 fold magnification) showing a mixture of blood samples, lysis buffers and PCR buffers before being amplified with emulsion PCR, in which the oil-encapsulated fluorescent dyes and nucleic acids are present in white portions, and gel-like materials and the background are present in black portions; and

FIG. 9 is a microscopic photograph (20 fold magnification) showing the mixture represented in FIG. 8 amplified with emulsion PCR, in which Target 1 represents DNA-probe-Cy3 with probe 1-Cy3 as labels, and Target 2 represents DNA-probe-Cy5 labeled with probe 1-Cy5. NC (positive control) represents the oil phase labeled with FAM dyes and WL represents the condition under white light.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

Commercially available kits for nucleic acid amplification, like Qiagen, primarily comprise two kinds of reagents, one for sample treatment and another for nucleic acid amplification. The reagent for sample treatment usually comprises protease K for digestion of proteins, a homogenizer solution for tissue sample homogenization, lysis buffers for tissue lysis, buffers for binding of nucleic acid molecules, washing buffers and elution buffers. On the other side, the reagent for nucleic acid amplification usually comprises a polymerase for nucleic acids elongation and amplification, deoxyribonucleotides (dNTPs) and the relevant buffers.

However, the operation requires several different tubes for a sample treatment and nucleic acid amplification. Thus, the risk for sample contamination is high and the operation period is long. In addition, toxic solvents like phenols or chloroform are used in the operation, which are not environmentally friendly. Furthermore, it is well-known that conventional nucleic acid isolation is restricted to a single type of sample and specific sample volumes and requires a long time for satisfactory purification results. In other words, the conventional nucleic acid isolation methods may not be applicable for treating several types of samples at one time or for treating much smaller or larger sample volumes.

To solve the existing problems, the inventors are providing a novel composition for biosample treatment and an efficient method for nucleic acid amplification which uses the composition. According to the disclosure, cell lysis and homogenization of the biosample can be completed in a single tube at one step, and the reagents for use in nucleic acid amplification can be added directly into the same tube without any further treatment beforehand. Thus, according to the disclosure, the process and operation period for sample treatment and nucleic acid amplification and the risk for sample contamination can be reduced. In addition, the background noises can be therefore reduced so that the sensitivity of the results can be increased. Specifically speaking, the operable sample volume can be enlarged to approximate 1˜30 μl, which meets current nucleic acid amplification requirements.

Specifically, one embodiment of the disclosure provides a composition for biosample treatment, which comprises at least one halocarbon, at least one polyether, and at least one surfactant.

The halocarbons disclosed herein may comprise fluorocarbons, chlorocarbons, bromocarbons or the like. Among these, perfluorocarbon is preferable in the composition according to the disclosure. Perfluorocarbons have been used as quenching liquids for electronic products and are widely used in the electronic industry. However, in developing novel compositions for sample treatments and nucleic acid amplification, the inventors have found that perfluorocarbons are capable of eliminating the interference of proteins and have chemical stability under high and low temperatures. In addition, no leftover perfluorocarbons remain in the final product after the treatment, thus, purity of the nucleic acids can be therefore improved.

In one embodiment of the disclosure, the perfluorocarbon may comprise C₁˜C₁₂ perfluoroalkane, such as tetrafluoromethane, hexafluoroethane, perfluropropane, perfluorobutane, perfluoropentane, perfluorohexane, perfluoroheptane or perfluorooctane; or C₃˜C₁₂ perfluorocycloalkane, such as perfluorocyclopropane, perfluorocyclobutane, perfluorocyclopentane, perfluorocyclohexane or perfluorocyclooctane.

In one embodiment of the disclosure, the polyether may comprise paraformaldehyde, polyoxymethylene, polyacetal, polyethylene glycol, polyethylene oxide, polyoxyethylene, polypropylene glycol, polypropylene oxide, polyoxypropylene, polytetramethylene glycol, polytetramethylene ether glycol, polytetrahydrofuran or a combination thereof, but it is not limited thereto.

In one embodiment of the disclosure, the surfactant can be any well-known surfactants for nucleic acid isolation without any specific restriction. Specific examples are sodium lauryl sulfate, lithium dodecyl sulfate, polysorbate, polyethylene glycol p-(1,1,3,3-tetramethylbutyl)-phenyl ether or a combination thereof.

In one embodiment of the disclosure, the composition for biosample treatment may contain 1˜70% by weight of the halocarbon based on the total weight of the composition. In one example, the composition for biosample treatment may contain 10˜60% by weight of the halocarbon based on the total weight of the composition. In another example, the composition for biosample treatment may contain 20˜50% by weight of the halocarbon based on the total weight of the composition.

In one embodiment, the composition for biosample treatment may contain 1˜50% by weight of the polyether based on the total weight of the composition. In one example, the composition for biosample treatment may contain 5˜45% by weight of the polyether based on the total weight of the composition. In one example, the composition for biosample treatment may contain 10˜40%, by weight of the polyether based on the total weight of the composition.

In one embodiment, the composition for biosample treatment may contain 0.01˜5% by weight of the surfactant based on the total weight of the composition. In one example, the composition for biosample treatment may contain 0.1˜4% by weight of the surfactant based on the total weight of the composition. In one example, the composition for biosample treatment may contain 1˜3%, by weight of the surfactant based on the total weight of the composition.

In one embodiment of the disclosure, the composition for biosample treatment may further comprise at least one reagent for nucleic acid amplification. The reagent for nucleic acid amplification is not specifically limited and may be self-formulated or commercially available. The reagent for nucleic acid amplification may comprise polymerases, deoxyribonucleotides, buffers or a combination thereof.

The ratio of the composition for biosample treatment and the reagent for nucleic acid amplification is not specifically limited. For reduced background noises to improve sensitivity, the ratio of the composition for biosample treatment and the reagent for nucleic acid amplification may be 1:1˜1:1000. In one example, the ratio of the composition for biosample treatment and the reagent for nucleic acid amplification may be 1:1˜1:500. In another example, the ratio of the composition for biosample treatment and the reagent for nucleic acid amplification may be 1:1˜1:200.

According to the disclosure, the nucleic acid amplification may comprise currently well-known reactions for nucleic acid amplification, such as a polymerase chain reaction (PCR), real-time polymerase chain reaction (real-time PCR), real-time quantitative polymerase chain reaction (real time quantitative PCR), multiplex polymerase chain reaction (multiplex PCR), multiplex quantitative polymerase chain reaction (multiplex quantitative PCR), reverse transcription polymerase chain reaction (RT-PCR), emulsion polymerase chain reaction (ePCR), solid polymerase chain reaction (solid PCR), quantitative reverse transcription polymerase chain reaction (qRT-PCR) or nucleic acid sequencing.

In one embodiment of the disclosure, the biosample for treatment is not specifically limited and may comprise cells, tissues, bloods, sera, urines, amniotic fluids, lymphatic fluids, saliva, feces, hairs, nails or a combination thereof.

In one embodiment of the disclosure, the nucleic acid for amplification may comprise single-stranded nucleic acids, double-stranded nucleic acids, nucleic acid fragments or a combination thereof.

According to the disclosure, the composition for biosample treatment makes biosamples homogenized and cells lysed in a single tube at one step, which is beneficial to a following process for nucleic acid amplification or the like.

One embodiment of the disclosure provides a method for nucleic acid amplification, comprising the following steps: mixing a composition and a biosample to form a homogenized solution, wherein the composition comprises at least one halocarbon, at least one polyether and at least one surfactant, mixing a reagent for nucleic acid amplification and the homogenized solution to form a mixture, and applying the mixture to nucleic acid amplification.

The halocarbon, polyether and surfactant have been defined above. The ratio of the halocarbon, polyether and surfactant in the composition has been defined above as well.

According to the disclosure, the process and operation period for the biosample treatment can be reduced. Also, due to less solvents used, the background noises can therefore be decreased. Thus, the treated biosample is applicable for several uses in the next step, such as nucleic acid amplification, nucleotide sequencing or the like. Highly-pure nucleic acids can therefore be obtained.

The reagent for nucleic acid amplification is not specifically limited and may be self-formulated reagents or commercially available reagents. The reagent for nucleic acid amplification may comprise polymerases, deoxyribonucleotides, buffers or a combination thereof.

The ratio of the composition for biosample treatment and the reagent for nucleic acid amplification is not specifically limited. For reduced background noises and improved sensitivity, the ratio of the composition for biosample treatment and the reagent for use in nucleic acid amplification may be 1:1˜1:1000. In one example, the ratio of the composition for biosample treatment and the reagent for use in nucleic acid amplification may be 1:1˜1:500. In another example, the ratio of the composition for biosample treatment and the reagent for use in nucleic acid amplification may be 1:1˜1:200.

According to the disclosure, the nucleic acid amplification may comprise currently well-known reactions for nucleic acid amplification, such as a polymerase chain reaction (PCR), real-time polymerase chain reaction (real-time PCR), real-time quantitative polymerase chain reaction (real time qPCR), multiplex polymerase chain reaction (multiplex PCR), multiplex quantitative polymerase chain reaction (multiplex quantitative PCR), reverse transcription polymerase chain reaction (RT-PCR), emulsion polymerase chain reaction (ePCR), solid polymerase chain reaction (solid PCR), quantitative reverse transcription polymerase chain reaction (qRT-PCR) or nucleic acid sequencing.

In one embodiment of the disclosure, the biosample for treatment is not specifically limited and may comprise cells, tissues, bloods, sera, urines, amniotic fluids, lymphatic fluids, saliva, feces, hairs, nails or a combination thereof.

In one embodiment of the disclosure, the nucleic acid for amplification may comprise single-stranded nucleic acids, double-stranded nucleic acids, nucleic acid fragments or a combination thereof.

According to the disclosure, the method for nucleic acid amplification can reduce the number of operation processes, operation periods and the background noises so as to obtain a clear result with high sensitivity by using the composition of the disclosure.

### EXAMPLE 1

### Single Tissue Type Treatment

(1) Nucleic Acid Purification by Using the Composition According to the Disclosure

(1-1) Preparation of Composition (1)

12.5 μl of perfluorohexane (Fluorinert™), 10 μl of polytetramethylene glycol and 0.1 μl of Triton X-100 were uniformly mixed to form Composition (1).

(1-2) Nucleic Acid Amplification of Mouse Bloods

10 μl of mouse bloods and 10 μl of mouse sera were separately added into Composition (1) and mixed uniformly. After a 3-minute static duration, the mixture was further mixed with 12.5 μl of the reagents for nucleic acid amplification (10 mM (NH₄)₂SO₄, 10 mM KCl, 2 mM MgSO₄, 20 mM of 0.1% Triton X-100, Tris-HCl, pH8.8, polymerases), in which and PCR amplification was processed with the following conditions.

(1-3) PCR Amplification

The target gene GAPDH was amplified according to the following conditions.

The mixtures treated above and 1mM of commercially available primer pairs (Fermentas, standard GAPDH primers) were added to the PCR machine (ABI 9700) for nucleic acid amplification according to the following PCR conditions:

Temperature 1: 95° C., for 15 minutes,

Cycle temperature: 95° C., for 10 seconds,
58° C., for 30 seconds, and
72° C., for 30 seconds,

Cycle numbers: 40 cycles;

Temperature 2: 72° C., 7 minutes.

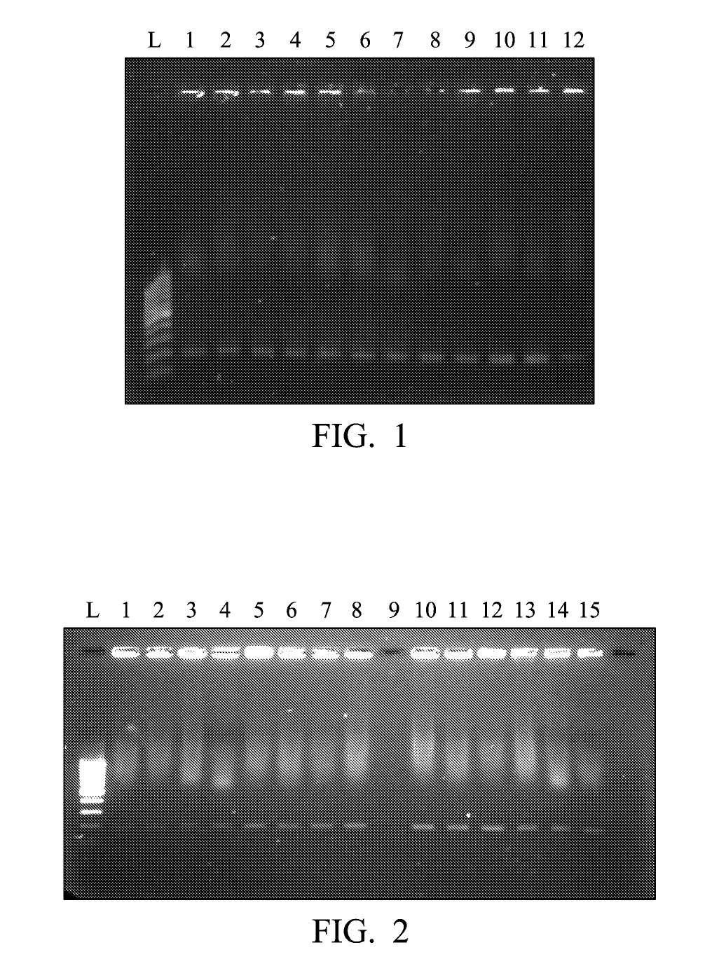

The amplified solution was run on an electrophoresis gel (TAE gel, 75V). The gel electrophoresis photograph is shown in FIG. 1.

(2) Nucleic Acid Amplification by Using Commercial Kits

(2-1) Nucleic Acid Amplification of Mouse Blood

10 μl of mouse bloods and 10 μl of mouse sera were separately treated with a Fermentas Mixima® reagent kit according to the protocol manual. Thereafter, the target gene GAPDH was amplified according to the following conditions.

(2-2) PCR Amplification

The target gene GAPDH was amplified according to the following conditions

The mixtures treated above and 1 mM of commercially available primer pairs (Fermentas Mixima® reagent kit, standard GAPDH primers) were added to the PCR machine (ABI 9700) for nucleic acid amplification according to the following PCR conditions:

Temperature 1: 95° C., for 15 minutes,

Cycle temperature: 95° C., for 10 seconds,
58° C., for 30 seconds, and
72° C., for 30 seconds,

Cycle numbers: 40 cycles;

Temperature 2: 72° C., 7 minutes.

The amplified solution was run on an electrophoresis gel (TAE gel, 75V). The results are shown in FIG. 1.

(3) Results Analyses

As shown in FIG. 1, Lane L shows a ladder marker, Lanes 1˜3 show the DNA sample from the mouse sera treated with the Fermentas Mixima® reagent kit, Lanes 4˜6 show the DNA sample from the mouse blood treated with the Fermentas Mixima® reagent kit, Lanes 7˜9 show the DNA sample from the mouse sera treated with Composition (1), and Lanes 10˜12 show the DNA sample from the mouse blood treated with Composition (1).

According to the results shown in FIG. 1, the DNA sample volume obtained from the mouse bloods and sera treated with Composition (1) was equivalent to that treated with the commercially available reagent kit.

Thus, it was realized that, the composition in one example of the disclosure was able to complete cell lysis and homogenization in a single tube at one step. In addition, the reagents for nucleic acid amplification were directly (without pre-treated) added into the same tube for subsequent amplification. Therefore, it was clear that use of the composition as disclosed herein, can simplify the operation process and reduce the risk of contamination and the operation period.

### EXAMPLES 2-5

### Treatment of Various Sample Volumes

Composition (1) obtained from Example 1 was well mixed with blood samples in equal volumes as listed in Table 1 and then respectively mixed with the reagents for nucleic acid amplification. The mixtures treated above and amplified with 1 mM of GAPDH primer pairs (Fermentas Mixima reagent kit, standard GAPDH primers) were added to the PCR machine (ABI 9700) for nucleic acid amplification. The PCR conditions were set as follows: denature at 95° C. and anneal at 58° C. in one cycle, and repeated for 40 cycles. The amplified solution was run on an electrophoresis gel (TAE gel, 75V). The results are shown in FIG. 2.

TABLE 1

| | | | Reagent for | The ratio of Composition |
|---|---|---|---|---|
| | Composition | Blood | nucleic acid | (1) in the total volume |
| Exam- | (1) | sample | amplification | of the treated mixture |
| ples | (μl) | (μl) | (μl) | (volume %) |
| 2 | 1 | 1 | 48 | 2 |
| 3 | 10 | 10 | 30 | 20 |
| 4 | 15 | 15 | 20 | 30 |
| 5 | 20 | 20 | 10 | 40 |

As shown in FIG. 2, Lane L shows a ladder marker, Lanes 1˜4 show the DNA sample obtained from Example 5, Lanes 5˜8 show the DNA sample obtained from Example 4, Lane 9 shows the blank; Lanes 10˜12 show the DNA sample obtained from Example 3, and Lanes 13˜15 show the DNA sample obtained from Example 2.

According to the results of Examples 2˜5, the composition in one example of the disclosure can be used for a large sample volume, meeting current nucleic acid amplification requirements.

### EXAMPLE 6

### Real-Time PCR Amplification

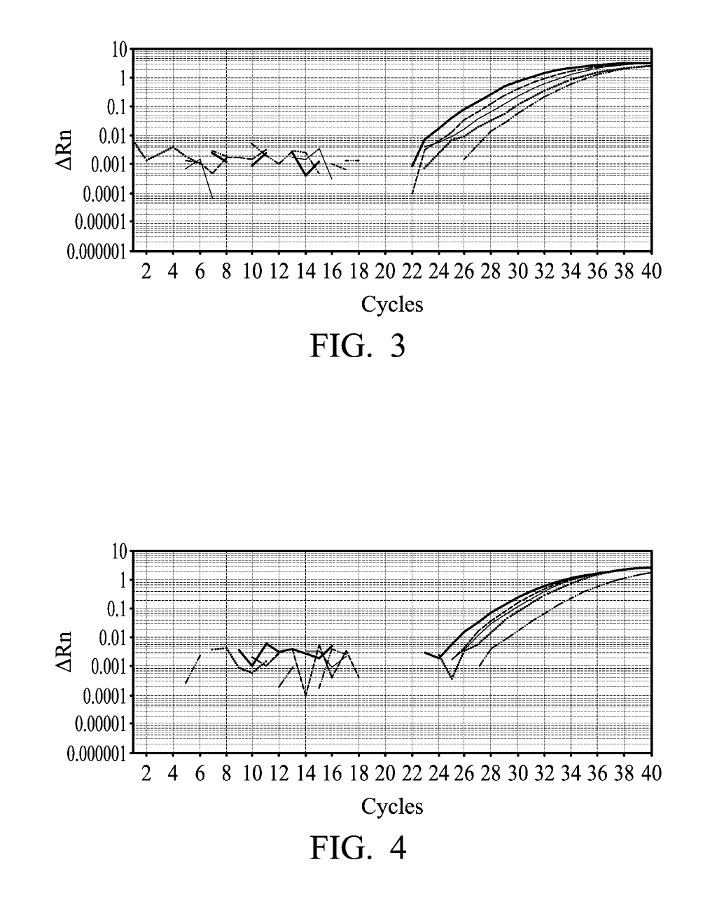

Composition (1) obtained from Example 1 was mixed with 10 μl of blood samples in equal volumes and then mixed with the reagents for nucleic acid amplification. The mixtures treated above and 1 mM of 18S/GAPDH primer pairs (ABI PCR control, standard 18S/GAPDH primers) were added to the PCR machine (ABI 9700) for nucleic acid amplification. The PCR conditions were set as follows: denature at 95° C. and anneal at 58° C. in one cycle, and repeated for 40 cycles. The results are shown in FIGS. 3 and 4, in which FIG. 3 shows PCR data of RNA 18S amplification by using ABI standard 18S primers and probes, and FIG. 4 shows PCR data of GAPDH amplification by using ABI standard 18S primers and probes.

According to the results, the composition in one example of the disclosure can be applied for real-time PCR, meeting current nucleic acid amplification requirements.

### EXAMPLE 7

### Virus Sample Treatment and Amplification

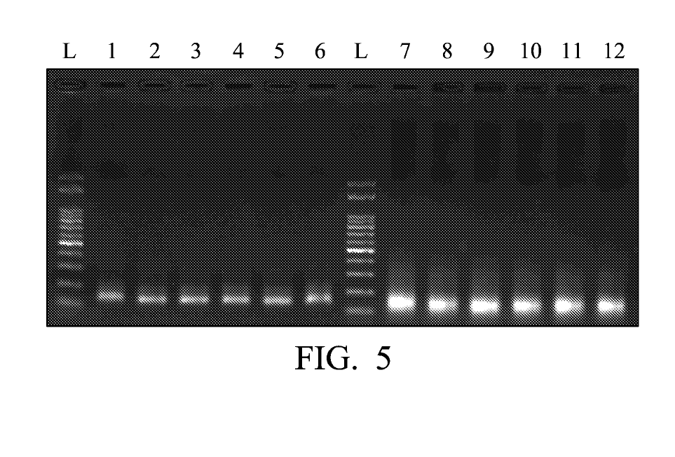

Composition (1) obtained from Example 1 was well-mixed with standard sera samples (Dengue virus) in equal volumes. After a 3-minute static duration, the mixture was further mixed with the reagents for nucleic acid amplification. Thereafter, the mixture was amplified with Dengue standard primer pairs (FDA standard primers) working on the PCR amplification machine (ABI 9700) to perform RT-PCR. The PCR conditions were set as follows: 95° C., for 15 minutes; 95° C., for 30 seconds, 60° C., for 30 seconds, and 72° C., for 30 seconds in one cycle and repeated for 40 cycles to amplify the RNA samples of the blood sample. The amplified solution was run on an electrophoresis gel (TAE gel, 75V). The results are shown in FIG. 5.

As shown in FIG. 5, Lane L shows a ladder marker, Lanes 1˜6 show the DNA sample treated with Composition (1), and Lanes 7˜12 show the DNA sample treated with Qiagen RT-PCR buffers. According to FIG. 5, the samples treated with the composition in one example of the invention after RT-PCR amplification showed clear bands on the electrophoresis gel. This clearly showed that the composition in one example of the disclosure can be applied for pathogens like viruses, meeting current nucleic acid amplification requirements.

### EXAMPLE 8

### Multiple PCR Amplification

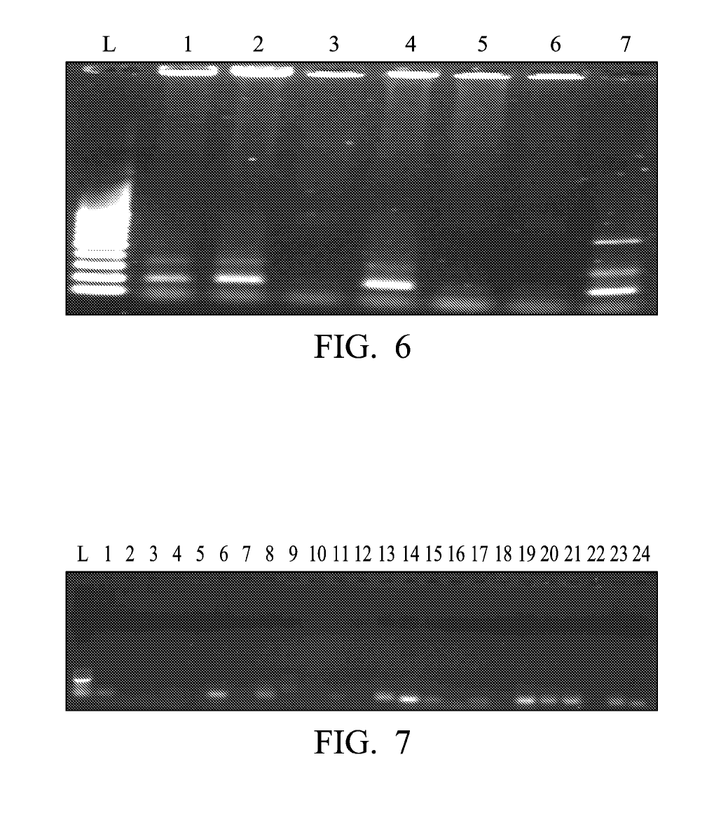

Composition (1) obtained from Example 1 was mixed with 5 μl of blood samples in equal volumes and then mixed with the reagents for nucleic acid amplification. The mixture treated above and 1 mM of GAPDH and β-actin primer pairs (ABI PCR control Primer Beta-actin 435234E, GAPDH 4308313) were added to the PCR machine (ABI 9700) for nucleic acid amplification. The PCR conditions were set as follows: 95° C., for 15 minutes, 95° C., for 10 seconds, 60° C., for 30 seconds, 70° C., for 30 seconds, and 72° C., for 30 seconds in one cycle and repeated for 35 cycles to amplify the DNA samples of the blood sample. The amplified solution was run on an electrophoresis gel (TAE gel, 75V). The results are shown in FIG. 6, in which ABI HUMAN control DNA 4312660 (10-3 μg) and sera sample (with no DNA) were taken as control.

As shown in FIG. 6, Lane L shows a ladder marker, Lane 1 shows the GADPH gene, Lane 2 shows β-actin-1 gene, Lane 3 shows the negative control blood sample, Lane 4 shows β-actin-2 gene and Lanes 5˜6 show the negative control blood sample. As a result, it is clear that the composition in one example of the disclosure can be applied to multiple gene amplification (like multiple PCR), meeting current nucleic acid amplification requirements.

### EXAMPLE 9

### Plant Tissues Treatment

25 μl of Composition (1) obtained from Example 1 was mixed with 1 mg of Semen Coicis, Vigna umbellate, Adenanthera pavonina, rice and brown rice, respectively. The mixture was further mixed with the reagents for nucleic acid amplification. Thereafter, the mixture was amplified with NCBI plant identification standard primers (EF1, E1F, 18S, UBQ, T, ACT2, AC11, TUA) on the PCR amplification machine (ABI 9700) to perform RT-PCR. The PCR conditions were set as follows: denature at 95° C. and anneal at 60° C. in one cycle and repeated for 40 cycles to amplify the DNA samples of the blood sample. The amplified solution was run on an electrophoresis gel (TAE gel, 75V). The results are shown in FIG. 7.

As shown in FIG. 7, Lane L shows a ladder marker, Lanes 1˜5 show the UBQ gene of Semen Coicis, Vigna umbellate, Adenanthera pavonina, rice and brown rice, respectively. Lanes 6˜10 show the UBQ10 gene of Semen Coicis, Vigna umbellate, Adenanthera pavonina, rice and brown rice, respectively. Lanes 11˜15 show the 25S rDNA of Semen Coicis, Vigna umbellate, Adenanthera pavonina, rice and brown rice, respectively. Lanes 16˜20 show the 18S rRNA of Semen Coicis, Vigna umbellate, Adenanthera pavonina, rice and brown rice, respectively. Lanes 21˜24 show the UBC gene of Semen Coicis, Vigna umbellate, Adenanthera pavonina, rice and brown rice, respectively. The results show that the composition in one example of the disclosure can be applied to nucleic acid amplification of plant tissues.

### EXAMPLE 11

### Broad Composite Ratio Range

Compositions (2)˜(10) were prepared according to the ratio listed in Table 2 below and used to sample treatment and nucleic acid amplification following the Steps (1-2) and (1-3) of Example 1.

TABLE 2

| | | Poly- | | | Reagent for | | |
|---|---|---|---|---|---|---|---|
| | Perfluoro- | tetramethylene | | | nucleic acid | Deionized | Perfluoro- |
| | hexane | glycol | Surfactant | Biosample | amplification | water | hexane |
| Compositions | (μl) | (μl) | (μl) | (μl) | (μl) | (μl) | (volume %) |
| (2) | 0.6 | 5 | 1 | 5 | 6 | 42.4 | 1 |
| (3) | 3 | 5 | 1 | 5 | 6 | 40 | 5 |
| (4) | 6 | 5 | 1 | 5 | 6 | 37 | 10 |
| (5) | 12 | 5 | 1 | 5 | 6 | 31 | 20 |
| (6) | 18 | 5 | 1 | 5 | 6 | 25 | 30 |
| (7) | 24 | 5 | 1 | 5 | 6 | 19 | 40 |
| (8) | 30 | 5 | 1 | 5 | 6 | 13 | 50 |
| (9) | 36 | 5 | 1 | 5 | 6 | 7 | 60 |
| (10) | 42 | 5 | 1 | 5 | 6 | 1 | 70 |

As shown in Table 2, the composition consisting of different composite ratios according to the disclosure can meet current nucleic acid amplification requirements.

### EXAMPLE 12

### Emulsion PCR Amplification

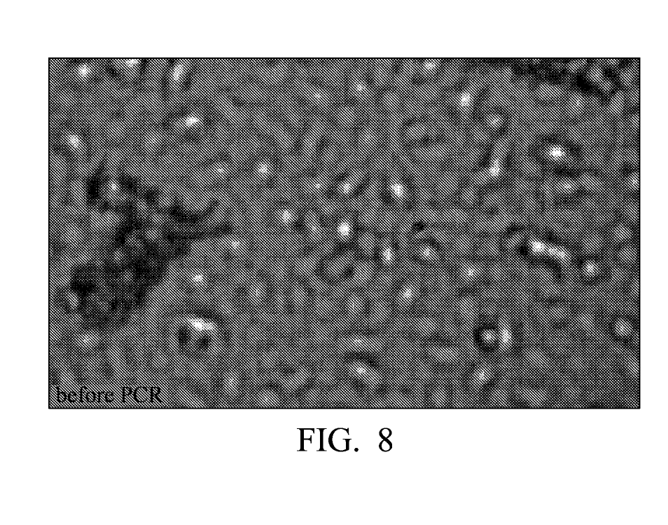

1. 10 μl of a lysis buffer (1 μl of perfluorohexane (Fluorinert™), 8 μl of Polytetramethylene glycol and 1 μl of Triton X-100) and PCR buffers were added to 20 μl of whole bloods and stood for about 3 minutes for the colors to change to green. The lysis buffer and PCR buffer can be added separately or pre-mixed before addition. 20 μl of oil (oil-surfactant and PCR components, the preparation listed below) was added to the treated sample and mixed at room temperature for 3 minutes. FIG. 8 shows a microscopic photograph (50 fold magnification), in which PCR reagents were encapsulated within the oil before processing emulsion PCR reaction. The white portion in FIG. 8 shows fluorescent dyes and encapsulated nucleic acids, while the black portion shows the background and gel-like materials.

The emulsion was then amplified with emulsion PCR under the conditions, 95° C., for 5 minutes, 94° C., for 45 seconds, 65° C., for 45 seconds, 55° C., for 45 seconds, 72° C., for 60 seconds in one cycle and repeated for 40 cycles. The solution was then kept at 72° C. for 5 minutes and stored at 4° C. for optical analyses.

2. The following components were thoroughly mixed in a 50 ml-centrifuge tube at 25° C. to prepare the oil-surfactant mixture.

| Components | Final concentration |
|---|---|
| Span 80 | 4.5% (vol/vol) |
| Tween 80 | 0.4% (vol/vol) |
| Triton X-100 | 0.05% (vol/vol) |
| Fluorescent dye (FAM dye) | 0.01% (vol/vol) |
| Mineral oil | to 1 ml (final volume) |

3. 400 μl of the oil-surfactant mixture was moved to CryoTube ampoules and 3×8 mm magnetic stir bars were added. The mixture was blended with the stir bars at 1,000 rpm.

4. The following components were mixed to form the water phase of emulsion:

| | |
|---|---|
| 10× Clone Pfu buffers | 1 μl |
| BSA (100 g/l) | 1 μl |
| Forward primers (10 μM) | 1 μl |
| Reverse primers (10 μM) | 1 μl |
| dNTPs (5 mM) | 2 μl |
| Pfu Turbo DNA polymerase | 1 μl |
| Probe 1-cy3 (100 nm) | 0.5 μl |
| Probe 2-cy5 (100 nm) | 0.5 μl |
| Template DNA | ≦10⁹ molecules (1.66 fmol) |
| Water | to 10 μl (final volume) |

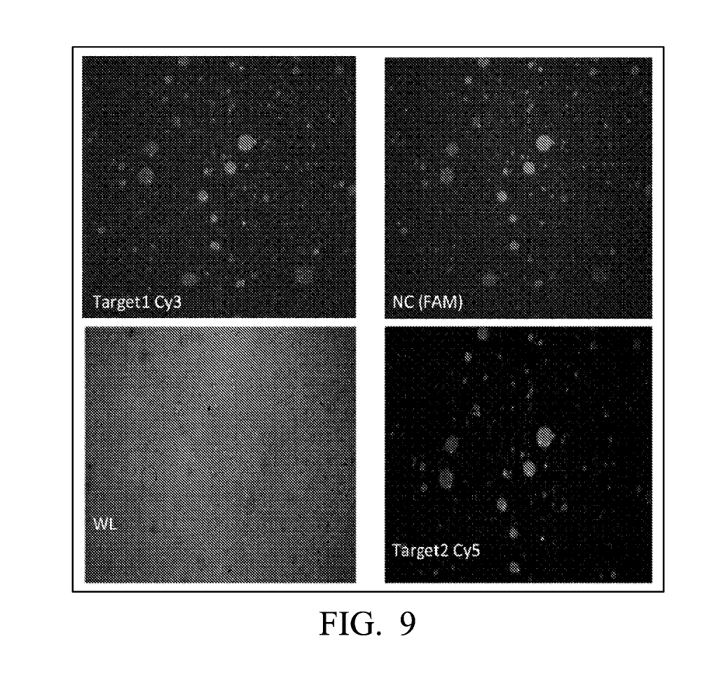

The first step can be processed randomly after the second, third or fourth step. The results are shown in FIG. 9 under 20 fold magnifications. In FIG. 10, Target 1 represents the DNA-probe-Cy3, in which the probe 1-Cy3 was labeled. Target 2 represents the DNA-probe-Cy5, in which the probe 2-Cy5 was labeled. NC (positive control) represents the oil phase labeled with FAM dyes. WL represents the condition under white light. The result showed that the biosample treated with the composition in one example of the disclosure resulted in complete oil-ball shapes after being amplified with emulsion PCR, which was beneficial for the next optical analyses.

According to the results of the example, it is clear that the composition of the disclosure is applicable for nucleic acid amplification in emulsions, meeting current nucleic acid amplification requirements.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A composition for a biosample treatment, comprising:
at least one halocarbon,
at least one polyether, and
at least one surfactant;
wherein the composition contains 1˜70% by weight of the halocarbon based on the total weight of the composition.

2. The composition as claimed in claim 1, wherein the halocarbon comprises perfluorocarbons.

3. The composition as claimed in claim 2, wherein the perfluorocarbon comprises tetrafluoromethane, hexafluoroethane, perfluropropane, perfluorobutane, perfluoropentane, perfluorohexane, perfluoroheptane or perfluorooctane.

4. The composition as claimed in claim 1, wherein the polyether comprises paraformaldehyde, polyoxymethylene, polyacetal, polyethylene glycol, polyethylene oxide, polyoxyethylene, polypropylene glycol, polypropylene oxide, polyoxypropylene, polytetramethylene glycol, polytetramethylene ether glycol, polytetrahydrofuran or a combination thereof.

5. The composition as claimed in claim 1,wherein the surfactant comprises odium lauryl sulfate, lithium dodecyl sulfate, polysorbate, polyethylene glycol p-(1,1,3,3-tetramethylbutyl)-phenyl ether or a combination thereof.

6. The composition as claimed in claim 1, wherein the composition contains 1˜50% by weight of the polyether based on the total weight of the composition.

7. The composition as claimed in claim 1, wherein the composition contains 0.01˜5% by weight of the surfactant based on the total weight of the composition.

8. The composition as claimed in claim 1, wherein the composition further comprises at least one reagent for nucleic acid amplification.

9. The composition as claimed in claim 8, wherein the reagent comprises polymerases, deoxyribonucleotides, buffers or a combination thereof.

10. The composition as claimed in claim 8, wherein a ratio of the composition and the reagent is 1:1 to 1:1000.

11. The composition as claimed in claim 8, wherein the nucleic acid amplification comprises a polymerase chain reaction (PCR), real time polymerase chain reaction (real time-PCR), real time quantitative polymerase chain reaction (real time quantitative PCR), multiplex polymerase chain reaction (multiplex PCR), reverse transcription polymerase chain reaction (RT-PCR), emulsion polymerase chain reaction (ePCR) or quantitative reverse transcription polymerase chain reaction (qRT-PCR).

12. The composition as claimed in claim 1, wherein the biosample comprises cells, tissues, bloods, sera, urines, amniotic fluids, lymphatic fluids, saliva, feces, hairs, nails or a combination thereof.

13. The composition as claimed in claim 1, wherein the nucleic acid comprises single-stranded nucleic acids, double-stranded nucleic acids, nucleic acid fragments or a combination thereof.

14. A method for nucleic acid amplification, comprising the following steps:
mixing a composition and a bios ample to form a homogenized solution, wherein the composition comprises at least one halocarbon, at least one polyether and at least one surfactant,
mixing a reagent for nucleic acid amplification and the homogenized solution to form a mixture, and
applying the mixture to nucleic acid amplification,
wherein the composition contains 1˜70% by weight of the halocarbon based on the total weight of the composition.

15. The method as claimed in claim 14, wherein the halocarbon comprises perfluorocarbons.

16. The method as claimed in claim 15, wherein the perfluorocarbon comprises tetrafluoromethane, hexafluoroethane, perfluropropane, perfluorobutane, perfluoropentane, perfluorohexane, perfluoroheptane or perfluorooctane.

17. The method as claimed in claim 14, wherein the polyether comprises paraformaldehyde, polyoxymethylene, polyacetal, polyethylene glycol, polyethylene oxide, polyoxyethylene, polypropylene glycol, polypropylene oxide, polyoxypropylene, polytetramethylene glycol, polytetramethylene ether glycol, polytetrahydrofuran or combination thereof.

18. The method as claimed in claim 14, wherein the surfactant comprises sodium lauryl sulfate, lithium dodecyl sulfate, polysorbate, polyethylene glycol p-(1,1,3,3-tetramethylbutyl)-phenyl ether or a combination thereof.

19. The method as claimed in claim 14, wherein the composition contains 1˜50% by weight of the polyether based on the total weight of the composition.

20. The method as claimed in claim 14, wherein the composition contains 0.01˜5% by weight of the surfactant based on the total weight of the composition.

21. The method as claimed in claim 14, wherein the reagent comprises polymerases, deoxyribonucleotides, buffers or a combination thereof.

22. The method as claimed in claim 14, wherein a ratio of the composition and the reagent is 1:1 to 1:1000.

23. The method as claimed in claim 14, wherein the nucleic acid amplification comprises a polymerase chain reaction (PCR), real time polymerase chain reaction (real time-PCR), real time quantitative polymerase chain reaction (real time quantitative PCR), multiplex polymerase chain reaction (multiplex PCR), reverse transcription polymerase chain reaction (RT-PCR), emulsion polymerase chain reaction (ePCR) or quantitative reverse transcription polymerase chain reaction (qRT-PCR).

24. The method as claimed in claim 14, wherein the biosample comprises cells, tissues, bloods, sera, urines, amniotic fluids, lymphatic fluids, saliva, feces, hairs, nails or a combination thereof.

25. The method as claimed in claim 14, wherein the nucleic acid comprises single-stranded nucleic acids, double-stranded nucleic acids, nucleic acid fragments or a combination thereof.
